(19) United States
(12) Patent Application Publication
(10) Pub. No.: US 2012/0142570 A1
(43) Pub. Date: Jun. 7, 2012

(54) HERBAL CLEANSING COMPOSITION AND METHOD OF PREPARING THE SAME

(75) Inventors: Ramesh Surianarayanan, Bangalore (IN); Krishan Manral, Bangalore (IN); Anindita Das, Bangalore (IN); Roseline Gandham, Bangalore (IN)

(73) Assignee: HIMALAYA GLOBAL HOLDINGS LTD., Dubai (AE)

(21) Appl. No.: 13/257,543

(22) PCT Filed: Mar. 20, 2010
(86) PCT No.: PCT/IB2010/000611

§ 371 Date: Feb. 15, 2012

(30) Foreign Application Priority Data
Mar. 20, 2009 (IN) 544/DEL/2009

(51) Int. Cl. A61K 8/97; A61Q 5/02; A61Q 19/10; A61K 8/73

(52) U.S. Cl. 510/121; 510/513; 510/463; 510/137

(57) ABSTRACT

Disclosed herein is a herbal cleansing composition comprising extracts of herbs along with dehydroxanthan gum and alkylpolyglucoside and cosmeceutically acceptable excipients and method of preparing the same.

### FIELD OF THE INVENTION

This invention, in general relates to a herbal cleansing composition. In more particularly, the present invention provides a herbal cleansing composition comprising herbs along with dehydroxanthan gum and alkylpolyglucoside and cosmeceutically acceptable excipients.

### BACKGROUND OF THE INVENTION

Sodium laureth sulfate, or sodium lauryl ether sulfate (SLES), is a cleansing agent and surfactant found in many personal care products. It is an inexpensive and very effective foaming agent.

The Cosmetic, Toiletry, and Fragrance Association (CTFA) and the American Cancer Society have stated that the common belief that SLES is a carcinogen is an urban legend, a view confirmed by toxicology research by the OSHA, NTP, and IARC. SLES and SLS, and subsequently the products containing them, have been found to contain parts-per-thousand to parts-per-million levels of 1,4-dioxane, with the recommendation that these levels be monitored. The U.S. Environmental Protection Agency considers 1,4-dioxane to be a probable human carcinogen, meaning that a daily consumption of one gram of 1,4-dioxane over a lifetime would increase the cancer risk by about one part in 3000.

Concern over the health risks, along with the probability that the ethoxylated surfactants in use contain a fair amount of residual 1,4-dioxane, has led many cosmetic and toiletry companies to examine the levels of dioxane in both their raw materials and their finished products.

In recent years, consumers are highly concerned with environmental safety, societal, and personal well-being impacts of products they buy as the environmental issues are increasing worldwide like global warming, decreasing rain forests, air pollution etc.

Alkyl Polyglucoside, which is a 100% natural, renewable, plant-derived surfactant, is a very strong tool to support “Greening” trend in Personal and Home Care Products. Because it is prepared from glucose and vegetable fatty alcohol and it shows higher performance as a main or co surfactant with excellent ecological property.

Alkyl Polyglucoside is very mild to human skin, and shows very good environmental compatibility and higher performance on every application in Personal and Home Care Products, Alkyl Polyglucoside can be regarded as the ideal “green” surfactants which add value to the formulated products and help to distinguish these products from conventional ones.

It is therefore an important aspect to develop a herbal cleansing formulation that is made of natural ingredients to provide better cleansing effect and is safe to use.

### RELATED ART

U.S. Patent application No. 20060286062 by Schep et al. discloses a natural hair shampoo or body wash or cleanser totally derived from plant materials to provide mild and non-irritating cleansing. The cleanser contains alkyl glucoside as a surfactant and a thickener that are from plant materials.

U.S. Pat. No. 5,925,615 by Kern et al. discloses a cleansing and conditioning shampoo composition that contains an extract of Zingiber zerumbet. The composition also contains water, surfactants, conditioning agents, thickening agents, stabilizers, preservatives, pH adjusting agents, fragrance, and color. The Methods of use and methods of making the shampoo composition are also disclosed in the art.

E.P. Patent application no. 1841402B9 by Lepilleur and Fruscella discloses a shampoo composition comprising a minced polygalactomannan hydrocolloid(s) in combination with a water soluble silicone compound. The hydrocolloids are derived from the endosperm of seeds of Tamarindus indica, Trigonella foenum-graecum, Cassia tora, Cassia obtusifolia, Ceratonia siliqua, Caesalpinia spinos, Cyamopsis tetragonoloba, and mixtures thereof.

### SUMMARY OF THE INVENTION

It is a principal objective of the present invention to provide a herbal cleansing composition comprising extracts of herbs along with alkyl polyglucosides and dehydroxanthan gum and cosmeceutically acceptable excipients, wherein said composition is devoid of any chemical contaminations.

It is another objective of the present invention to provide a herbal cleansing composition comprising extracts of herbs along with alkylpolyglucosides and dehydroxanthan gum and cosmeceutically acceptable excipients, wherein said composition provides superior cleansing hair and skin benefits.

It is yet another objective of the present invention to provide a herbal cleansing composition comprising extracts of, herbs, along with alkylpolyglucosides and dehydroxanthan gum and cosmeceutically acceptable excipients, wherein said compositions are made using emulsifiers that are made from natural ingredients.

Further objective of the present invention is to provide a method of preparing a cleansing composition comprising extracts of herbs, along with alkylpolyglucosides and dehydroxanthan gum and cosmeceutically acceptable excipients, wherein said method is devoid of using any solvent.

The above and the other objects of the present invention are attended according to following preferred embodiments of the present invention, however the scope of the invention is not restricted to the particular embodiment.

In accordance with one embodiment of the present invention, there is provided a herbal cleansing composition comprising extracts of herbs along with alkylpolyglucosides and dehydroxanthan gum and cosmeceutically acceptable excipients, wherein the extract is prepared employing a super critical fluid extraction and/or water extraction.

In accordance with another embodiment of the present invention, there is provided a herbal cleansing composition comprising extracts of herbs along with alkylpolyglucosides, dehydroxanthan gum and cosmeceutically acceptable excipients, wherein the extract of herbs is obtained from a blend of herbs selected from Oryza sativa, Cicer arietinum, Hibiscus rosa-sinensis, Vetiver zizanioides, Cynodon dactylon, Cyperus rotundas, Sida cordifolia, Glycyrrhiza glabra Aegle marmelos, Myristica fragrans, Carica papaya, Ananas sativus Melia azadirachta, Curcuma longa, Terminalia chebula, Terminalia bellerica, Phyllanthus emblica, Ficus racemosa, Ficus bengalensis, Ficus religiosa or in various combinations thereof.

In accordance with one other embodiment of the present invention, there is provided a herbal cleansing composition comprising extracts of herbs along with alkylpolyglucosides, dehydroxanthan gum and cosmeceutically acceptable excipients, wherein the blend is used in the range of 0.0001% to 20.00%.

In accordance with one other embodiment of the present invention, there is provided a herbal cleansing composition comprising extracts of herbs along with alkylpolyglucosides, dehydroxanthan gum and cosmeceutically acceptable excipients, wherein said composition is formulated in various kind of cleansing formulation preferably shampoos and face wash formulation.

In accordance with yet another embodiment of the present invention there is provided a herbal cleansing composition comprising extracts of herbs along with alkylpolyglucosides, dehydroxanthan gum and cosmeceutically acceptable excipients, wherein shampoo is Gentle Baby Shampoo and face washes are revitalising body wash, exfoliating body wash and purifying body wash.

In accordance with yet another embodiment of the present invention, there is provided a herbal cleansing composition comprising extracts of herbs along with alkylpolyglucosides, dehydroxanthan gum and cosmeceutically acceptable excipients, wherein the cleansing composition is revitalizing body wash and wherein said herbs are selected from Vetiveria zizanoides, Cynodon dactylon, Cyperus rotundus, Sida cordifolia, Glycyrrhiza glabra.

In accordance with yet another embodiment of the present invention, there is provided a herbal cleansing composition comprising extracts of herbs along with alkylpolyglucosides, dehydroxanthan gum and cosmeceutically acceptable excipients, wherein, the cleansing composition is exfoliating body wash and wherein said herbs are selected from Aegle marmelos, Myristica fragrans, Carica papaya, and Ananas sativus and in combinations thereof.

In accordance with yet another embodiment of the present invention, there is provided a herbal cleansing composition comprising extracts of herbs along with alkylpolyglucosides, dehydroxanthan gum and cosmeceutically acceptable excipients, wherein, the cleansing composition is purifying body wash and wherein said herbs are selected from Melia azadirachta, Curcuma longa, Terminalia chebula, Terminalia bellerica, Phyllanthus emblica, Ficus racemosa, Ficus bengalensis and Ficus religiosa and in combinations thereof.

In accordance with yet another embodiment of the present invention, there is provided a method to prepare herbal cleansing composition, wherein said method comprising blending the herbs and extracting the resultant herbal blend employing Super Critical Fluid Extraction (SCFE), mixing the resultant extract of blend with dehydroxanthan gum and alkylpolyglucoside and cosmeceutically acceptable excipients.

In accordance with yet another embodiment of the present invention, there is provided a method to prepare herbal cleansing composition, wherein said method comprising blending the herbs and extracting the resultant herbal blend employing percolation or soxhalation using water, mixing the resultant extract of blend with dehydroxanthan gum and alkylpolyglucoside and cosmeceutically acceptable excipients.

### DETAILED DESCRIPTION OF THE INVENTION

While this specification concludes with claims particularly pointing out and distinctly claiming that, which is regarded as the invention, it is anticipated that the invention can be more readily understood through reading the following detailed description of the invention and study of the included examples.

The present invention describes about various types of herbal cleansing composition such as shampoos and face wash shampoos. The preparation comprising various herbs extracts along with alkylpolyglucosideS, dehydroxanthan gum as a synergistic combination for superior cleansing hair and skin benefits.

In accordance with the present in invention, said extract of herbs can be prepared by using any parts of herbs, preferably grains of Oryza sativa, seeds of Cicer arietinum, flowers of Hibiscus rosa-sinensis, roots of Vetiver zizanioides, whole parts of Cynodon dactylon, roots of Cyperus rotundus, roots of Sida cordifolia, roots of Glycyrrhiza glabra, fruits of Aegle marmelos, nutmeg of Myristica fragrans, fruits of Carica papaya, fruits of Ananas sativus, leaves of Melia azadirachta, leaves of Curcuma longa, roots of Terminalia chebula, fruits of Terminalia bellerica, fruits of Phyllanthus emblica, fruits of Ficus racemosa, bark of Ficus bengalensis, bark of Ficus religiosa.

In accordance with another preferred embodiment, there is provided a process for preparation of extract from said herbs, wherein process is performed using suitable extraction technique, preferably super critical fluid extraction, cold percolation method, hot-soxhlation method or percolation method.

The following non-limiting examples illustrate in details about the invention.

However, they are, not intended to be limiting the scope of present invention in any way.

### Example-1

TABLE 1
Cleansing formula

| | Use of the | Formula A | Formula B | Formula C |
|---|---|---|---|---|
| Ingredients | material | % addn. | % addn. | % addn. |
| Dehydro xanthan | Thickener | 0.00 | 0.00 | 0.85 |
| gum | | | | |
| Xanthan gum | Thickener | 0.00 | 0.85 | 0.00 |
| Guar gum | Thickener | 0.85 | 0.00 | 0.00 |
| Glycerin | Humectant | 3.00 | 3.00 | 3.00 |
| Coco glucoside | Foaming agent | 40.00 | 40.00 | 40.00 |
| Decyl glucoside | Foaming agent | 20.00 | 20.00 | 20.00 |
| Coco glucoside & | Foam stabilizer | 5.00 | 5.00 | 5.00 |
| Glyceryl oleate | | | | |
| Citric acid | pH neutralizer | 0.60 | 0.60 | 0.60 |
| Natural Vitamin E | Anti-oxidant | 0.10 | 0.10 | 0.10 |
| Potassium sorbate | Preservative | 0.50 | 0.50 | 0.50 |
| Fragrance | Fragrance | 0.50 | 0.50 | 0.50 |
| DM Water | Diluent | 29.45 | 29.45 | 29.45 |
| Total | | 100.00 | 100.00 | 100.00 |

Manufacturing Method

1. Decyl Glucoside, Coco Glucoside and Coco glucoside & Glyceryl oleate, is weighed in the side vessel and mixed Well for 10 minutes. The mixture pH is adjusted by 20% citric acid solution around 5.50.
2. Glycerin is taken in a separate SS Vessel and Amaze-XT/Xanthan Gum/Guar Gum is dispersed in it using Karl Homogeniser.
3. DM water is taken in the main vessel and to it is added the slurry of glycerine & gum, to form gum dispersion.
4. The surfactant mixture from the step 1 is then transferred to the main vessel under vacuum. This is mixed for 20 minutes to ensure complete mixing.
5. Natural Vitamin E is added into the main vessel and mixed well.
6. Fragrance is added in the mixing vessel and mixed well.
7. Finally, Potassium Sorbate is added to the mail vessel and mixed well.
8. DM water is added finally to adjust the batch quantity to 100%.

### Example-2

### Evaluation of Cleansing Formula

1. All the three formulations analyzed for viscosity evaluation in Brookefield Viscometer DV II+ Model (USA), and the results were found as follows,

TABLE 2

| | | Viscosity | |
|---|---|---|---|
| S. | FORMULA | (at 30° C., LV-4 | |
| NO. | NO. | Spindle at 50 rpm) | Appearance of product |
| 1 | A | 311 cps | Very Thin product |
| 2 | B | 6559 cps | Very thick & lumpy product |
| 3 | C | 5099 cps | Flowing product of optimum |
| | | | viscosity |

Results:
The formula C, which is with 0.85% Amaze-XT excels out Formula A and Formula B with Guar Gum & Xanthan Gum, respectively.

### Example-3

TABLE 3
Formulation of Cleansing composition with Dehydro Xanthan gum

| | | Formula A |
|---|---|---|
| Ingredients | Use of the material | % addn. |
| Dehydro xanthan gum | Thickener | 0.85 |
| Glycerine | Humectant | 3.00 |
| Coco glucoside | Foaming agent | 40.00 |
| Decyl glucoside | Foaming agent | 20.00 |
| Coco glucoside & Glyceryl | Foam stabilizer | 5.00 |
| oleate | | |
| Citric acid | pH neutralizer | 0.60 |
| Natural Vitamin E | Anti-oxidant | 0.10 |
| Potassium sorbate | Preservative | 0.50 |
| Fragrance | Fragrance | 0.50 |
| DM Water | Diluent | 29.45 |
| Total | | 100.00 |

Manufacturing Method

1. Decyl glucoside, coco glucoside and Coco glucoside & Glyceryl oleate is weighed in the main vessel and mixed well for 10 minutes. The mixture pH is adjusted by 20% citric acid solution around 5.50.
2. A portion of DM water is weighed in a vessel and kept in a propeller stirrer. Dehydro xanthan gum is wet in glycerin and transferred into the DM water slowly. This is mixed for 20 minutes to ensure complete mixing. This is transferred into main vessel and mixed for 20 minutes.
3. Potassium sorbate is dissolved in a small portion of water.
4. Natural Vitamin E is added into the main vessel and mixed well.
5. Fragrance is added into the main vessel and mixed well.
6. DM water is added finally to adjust the batch quantity to 100%.

Example Formulations:

Based on studies the following are the examples of different cleanser products with alkylpolyglucoside and dehydroxanthan gum which includes gentle baby shampoo, Revitalizing Body Wash, Exfoliating Body Wash and Purifying Body Wash.

### Example-4

TABLE 3
Formulation Details of Gentle Baby Shampoo

| Phase | Ingredients | Percentage addition |
|---|---|---|
| A | Plantacare 818 UP | 10.00 |
| | Lamesoft PO 65 | 1.00 |
| B | Citric Acid I.P | 0.20 |
| | DM Water | 3.00 |
| C | Glycerin I.P | 5.00 |
| | Amaze-XT (Dehydroxanthum | 0.65-2.00 |
| | gum) | |
| D | DM Water | 64.15 |
| E | Oryza sativa extract, Cicer | 1.00-20.00 |
| | arietinum extract, Hibiscus rosa- | |
| | sinensis extract, Vetiver | |
| | zizanioides extract (Herbal blend | |
| | coded as EGBS-09) | |
| F | Benzyl Alcohol | 0.30 |
| | Unicat MM | 0.10 |
| H | Perfume | 0.50 |
| | Plantacare 818 UP | 5.00 |
| | Plantacare 2000 UP | 5.00 |
| Total | | 100.00 |

Method of Manufacturing:

1. Charge DM Water in a side SS vessel and heat the entire contents to 95° C., boil it for 30 minutes, start chiller, bring down temperature to 25° C., and dispense the total amount of boiled & cooled DM Water (67.150%) required for the batch into a separate SS vessel and keep it covered. Keep DM Water (2.00%) for rinsing separately in covered SS vessel.
2. Take boiled & cooled DM Water 3.00%) in a side vessel, add Citric Acid into it, and mix well to form ‘Phase B’.
3. Charge ‘Phase A’ ingredients in a side SS vessel and mix for 5 minutes with continuous stirring at 20 rpm. Stop mixing and heat the entire contents to 60° C.
4. Add pre-mixed ‘Phase B’ to the main vessel in part-by-part quantity. Continue mixing at 10 rpm for 5 minutes after addition of each part of Citric Acid Solution. Preferably, add it in four parts. Finally mix it for 5 minutes at 10 rpm. Stop mixing and maintain temperature at 60° C. for 30-40 mts till foam disappears completely. This is ‘Phase AB’.
5. Charge the freshly boiled and cooled ‘Phase D’ DM Water into the main vessel and continue mixing at 20 rpm.
6. Take ‘Phase C’ Glycerin in a side SS vessel and disperse ‘Amaze-XT’ into it by using Karl Homogenizer to form smooth slurry. Scrap occasionally to remove any lumps.
7. Add the pre-mixed slurry of Glycerin and Amaze-XT into the vortex of the water in the main vessel to form ‘Phase CD’. Mix it at a very high speed of 80 rpm for 5 minutes, scrap the sides of the vessel and anchor blades, and again mix at 80 rpm for 10 minutes.
8. Transfer the ‘Phase E’ active in a side SS vessel, add DM Water (4.000%) into it, mix well and add to main vessel containing Amaze-XT gel, by passing it through nylon muslin filter to form ‘Phase CDE’. Continue mixing at 60 rpm for 20 minutes to form a uniform gel till there are no lumps found in the solution. Scrap the sides of the vessel and anchor blades, and again mix at 30 rpm for 10 minutes.
9. Start Vacuum at 450-550 mm of Hg with mixing at 30 rpm for 60 minutes till no lumps remain and air bubbles subside to minimum.
10. Under vacuum, charge the ‘Phase AB’ into the main vessel containing ‘Phase CDE’, from bottom and mix with continuous stirring at 10 rpm till the complete transfer take place, to form ‘Phase ABCDE’.
11. Take out 5 Kg of bulk from below from the main vessel in a side SS vessel, add the Phase F′ Benzyl Alcohol into it, mix well with SS Spatula and transfer the entire contents into the main vessel to form ‘Phase ABCDEF’ and mix for 10 minutes at 20 rpm.
12. Add ‘Phase G’ Unicat MM to the main vessel and mix at 20 rpm for 10 mts to form ‘Phase ABCDEFG’.
13. Apply Vacuum at 450-550 mm of Hg with mixing at 10 rpm for 60 minutes till air bubbles subside to minimum.
14. Charge ‘Phase H’ ingredients in a side SS vessel and mix for 10 minutes with continuous stirring at 20 rpm till it becomes clear.
15. Under vacuum, transfer the pre-mixed ‘Phase H’ to the main vessel from bottom and mix with continuous stirring at 10 rpm till the complete transfer take place to form ‘Phase ABCDEFGH’,
16. Release vacuum and mix the entire contents for 20 minutes at 20 rpm.
17. Check the pH of the product, which should be in the range of pH=6.0-7.0.

### Example-5

TABLE 4
Formulation Details of Revitalizing Body Wash

| Phase | Ingredients | Percentage addition |
|---|---|---|
| A | Plantacare 818 UP | 24.500 |
| | Plantacare 2000 UP | 13.000 |
| | Lamesoft PO 65 | 2.000 |
| B | Citric Acid I.P | 0.921 |
| | DM Water | 3.000 |
| C | Glycerin I.P | 3.000 |
| | Amaze-XT | 0.7000-2.000 |
| | (Dehydroxanthan gum) | |
| D | DM Water | 47.929 |
| E | Potassium Sorbate | 0.500 |
| | DM Water | 1.000 |
| F | Vetiveria zizanoides | 1.000-20% |
| | Extract, Cynodon | |
| | dactylon whole plant | |
| | extract, Cyperus | |
| | rotundus root extract, | |
| | sida cordifolia root | |
| | extract, Glycyrrhiza | |
| | glabra (Licorice) Root | |
| | Extract (herbal blend | |
| | coded as ERBW-18) | |
| | Plantacare 818 UP | 0.500 |
| G | Perfume | 0.500 |
| | Natural Vitamin E | 0.100 |
| H | Biovert Substrate | 1.000 |
| | Solution | |
| | Biovert Enzyme | 0.050 |
| | Solution | |
| Total | | 100.000 |

### Example-6

### Method of Manufacturing

1, Charge DM Water in a side SS vessel and heat the entire contents to 95° C., boil it for 30 minutes, start chiller, bring down temperature to 25° C., and dispense the total amount of boiled & cooled DM Water (47.929%) required for the batch into a separate SS vessel and keep it covered. Keep DM Water (2.00%) for rinsing separately in covered SS vessel.
2. Take boiled & cooled DM Water (MO %) in a side vessel, add Citric Acid into it, and mix well to form ‘Phase B’.
3. Charge ‘Phase A’ ingredients in a side SS vessel. Add pre-mixed ‘Phase B’, mix for 5 minutes with continuous stirring at 20 rpm. Stop mixing and heat the entire contents to 70° C., start chiller, bring down temperature to 60° C., maintain at same temperature for 1-2 hrs, till foam disappears completely. Again start chiller and bring down temperature to 35° C. This is ‘Phase AB’.
4. Charge the freshly boiled and cooled ‘Phase D’ DM Water into the main vessel and continue mixing at 20 rpm.
5, Take ‘Phase C’ Glycerin′ in a side SS vessel and disperse ‘Amaze-XT’ into it by using Karl Homogenizer to form smooth slurry. Soap occasionally to remove any lumps.
6. Add the pre-mixed slurry of Glycerin and Amaze-XT into the vortex of the water in the main vessel to form ‘Phase CD’. Mix it at very high speed of 80 rpm for 5 minutes, scrap the sides of the vessel and anchor blades, and again mix at 80 rpm for 10 minutes.
7. Charge the ‘Phase E’ Potassium Sorbate and boiled & cooled DM water (1.00%) into the side vessel and mix to form a uniform solution.
8. Transfer ‘Phase E’ into main vessel containing Amaze-XT gel to form ‘Phase CDE’, and continue mixing at 60 rpm for 20 minutes to form a uniform gel till there is no lumps found in the solution. Scrap the sides of the vessel and anchor blades, and again mix at 30 rpm for 10 minutes.
9. Start Vacuum at 450-550 mm of Hg with mixing at 30 rpm for 60 minutes till no lumps remain and air bubbles subside to minimum.
10. Under vacuum charge the ‘Phase AB’ into the main vessel containing ‘Phase CDE’, from bottom and mix with continuous stirring at 10 rpm till the complete transfer take place, to form ‘Phase ABCDE’.
11. Charge the ‘Phase F’ Active, in a stainless steel container and mix with Plantacare 818 UP to form slurry.
12. Release vacuum and transfer the pre-mixed ‘Phase F’ to the main vessel by passing it through nylon muslin filter with continuous stirring at 20 rpm for 10 minutes to form ‘Phase ABCDEF’.
13. Give Vacuum at 550 mm of Hg for 60 minutes with continuous mixing at 20 rpm, till all the air bubbles are removed.
14. Charge the ‘Phase G’ ingredients into the side SS vessel and mix for 5 minutes to form a uniform blend of Perfume & Natural Vitamin E.
15. Release vacuum and transfer the pre-mixed ‘Phase G’ into the main vessel to form ‘Phase ABCDEFG’ and mix for 20 minutes.
16. Charge the ‘Phase H’ ingredient, Biovert Substrate Solution to the main vessel and mix with continuous stirring for 5 minutes.
17. Take out 5 Kg of bulk from below from the main vessel in a side SS vessel, add the ‘Phase H’ ingredient, Biovert Enzyme Solution into it, mix well with SS Spatula and transfer the entire contents into the main vessel to form ‘Phase ABCDEFGH’ and mix for 20 minutes.
18. Check the pH of the product, which should be in the range of pH=4.5-5.4.
19. Submit the sample to QA Dept, for analysis.
20. Fill the product after QA Dept.'s approval.

### Example-7

TABLE 5
Formulation Details of Exfoliating Body Wash

| Phase | Ingredients | Percentage addition |
|---|---|---|
| A | Plantacare 818 UP | 24.500 |
| | Plantacare 2000 UP | 13.000 |
| | Lamesoft PO 65 | 2.000 |
| B | Citric Acid I.P | 0.921 |
| | DM Water | 3.000 |
| C | Glycerin I.P | 3.000 |
| | Dehydroxanthan gum | 0.750-2.000 |
| D | DM Water | 47.679 |
| E | Potassium Sorbate | 0.500 |
| | DM Water | 1.000 |
| F | Aegle marmelos Fruit | 1.00-20.00% |
| | Extract, Myristica fragrans | |
| | (Nutmeg) Extract, Carica | |
| | papaya (Papaya) Fruit | |
| | Powder, Ananas sativus | |
| | (Pineapple) Fruit powder | |
| | (Herbal blend coded as | |
| | EEBW-27) | |
| | Plantacare 818 UP | 0.500 |
| G | Organic Nutmeg Shell | 0.050 |
| | Powder | |
| | Natural Walnut Shell | 0.100 |
| | Powder | |
| | Natural Woodapple Shell | 0.100 |
| | Powder | |
| H | Perfume | 0.500 |
| | Natural Vitamin E | 0.100 |
| I | Biovert Substrate Solution | 1.000 |
| | Biovert Enzyme Solution | 0.050 |
| Total | | 100 |

Method of Manufacturing:

1. Charge DM Water in a side SS vessel and heat the entire contents to 95° C., boil it for 30 minutes, start chiller, bring down temperature to 25° C., and dispense the total amount of boiled & cooled DM Water (47.679%) required for the batch into a separate SS vessel and keep it covered. Keep DM Water (2.00%) for rinsing separately in covered SS vessel.
2. Take boiled & cooled DM Water (3.00%) in a side vessel, add Citric Acid into it, and mix well to form ‘Phase B’
3. Charge ‘Phase A’ ingredients in a side SS vessel. Add pre-mixed ‘Phase B’ mix for 5 minutes with continuous stirring at 20 rpm, Stop mixing and heat the entire contents to 70° C., start chiller, bring down temperature to 60° C., maintain at same temperature for 1-2 hrs, till foam disappears completely. Again start chiller and bring down temperature to 35° C. This is ‘Phase AB’.
4. Charge the freshly boiled and cooled ‘Phase D’ DM Water into the main vessel and continue mixing at 20 rpm.
5, Take ‘Phase C’ Glycerin′ in a side SS vessel and disperse ‘Amaze-XT’ into it by using Karl Homogenizer to form smooth slurry. Scrap occasionally to remove any lumps.
6. Add the pre-mixed slurry of Glycerin and Amaze-XT into the vortex of the water in the main vessel to form ‘Phase CD’. Mix it at very high speed of 80 rpm for 5 minutes, scrap the sides of the vessel and anchor blades, and again mix at 80 rpm for 10 minutes.
7. Charge the ‘Phase E’ Potassium Sorbate and boiled & cooled DM water (1.00%) into the side vessel and mix to form a uniform solution.
8. Transfer ‘Phase E’ into main vessel containing Amaze-XT gel to form ‘Phase CDE’, and continue mixing at 60 rpm for 20 minutes to form a uniform gel till there are no lumps found in the solution. Scrap the sides of the vessel and anchor blades, and again mix at 30 rpm for 10 minutes.
9. Start Vacuum at 450-550 mm of Hg with mixing at 30 rpm for 60 minutes till no lumps remain and air bubbles subside to minimum.
10. Under vacuum charge the ‘Phase AB’ into the main vessel containing ‘Phase CDE’, from bottom and mix with continuous stirring at 10 rpm till the complete transfer take place, to form ‘Phase ABCDE’.
11. Charge the ‘Phase F’ Active, Organic ARD-434 in a stainless steel container and mix with Plantacare 818 UP to form slurry.
12. Release vacuum and transfer the pre-mixed ‘Phase F’ to the main vessel by passing it through nylon muslin filter with continuous stirring at 20 rpm for 10 minutes to form ‘Phase ABCDEF’.
13. Give Vacuum at 550 mm of Hg for 60 minutes with continuous mixing at 20 rpm, till all the air bubbles are removed.
14. Charge the ‘Phase G’ ingredient, Natural Wood Apple Shell Powder, Natural Walnut Shell Powder and Organic Nutmeg Shell Powder (unsterilized), for steam sterilization at a temperature of 121° C., and a pressure of 1.2 Kg/cm², for a time period of about 20-30 minutes. Cool it to room temperature.
15. Charge the ‘Phase G’ ingredient (immediately after steam sterilization), into the main vessel to form ‘Phase ABCDEFG’ and mix with continuous stirring at 20 rpm for 10 minutes.
16. Charge the ‘Phase H’ ingredients into the side SS vessel and mix for 5 minutes to form a uniform blend of Perfume & Natural Vitamin E.
17. Release vacuum and transfer the pre-mixed ‘Phase H’ into the main vessel to form ‘Phase ABCDEFGH’ and mix for 20 minutes.
18. Charge the ‘Phase I’ ingredient, Biovert Substrate Solution to the main vessel and mix with continuous stirring for 5 minutes.
19. Take out 5 Kg of bulk from below from the main vessel in a side SS vessel, add the ‘Phase I’ ingredient, Biovert Enzyme Solution into it, mix well with SS Spatula and transfer the entire contents into the main vessel to form ‘Phase ABCDEFGH’ and mix for 20 minutes.
20. Check the pH of the product, which should be in the range of pH=4.5-5.4.

### Example-8

TABLE 6
Formulation Details Purifying Body Wash

| Phase | Ingredients | Percentage addition |
|---|---|---|
| A | Plantacare 818 UP | 24.500 |
| | Plantacare 2000 UP | 13.000 |
| | Lamesoft PO 65 | 2.000 |
| B | Citric Acid I.P | 0.921 |
| | DM Water | 3.000 |
| C | Glycerin I.P | 3.000 |
| | Amaze-XT | 0.650-1.000 |
| D | DM Water | 47.929 |
| E | Potassium Sorbate | 0.500 |
| | DM Water | 1.000 |
| F | Melia azadirachta Leaf | 1.00-20.00% |
| | Extract, Curcuma longa | |
| | (Turmeric) root Extract, | |
| | Terminalia chebula | |
| | Fruit Extract, | |
| | Terminalid bellerica | |
| | Fruit Extract, | |
| | Phyllanthus emblica | |
| | Fruit Extract, Ficus | |
| | racemosa Bark | |
| | Extract*, Ficus | |
| | bengalensis Bark | |
| | Extract, Ficus religiosa | |
| | Bark Extract (Herbal | |
| | blend coded as EPBW- | |
| | 36) | |
| | Plantacare 818 UP | 0.500 |
| G | Perfume | 0.500 |
| | Natural Vitamin E | 0.100 |
| H | Biovert Substrate | 1.000 |
| | Solution | |
| | Biovert Enzyme | 0.050 |
| | Solution | |
| Total | | 100.000 |

Method of Manufacturing:

1. Charge DM Water in a side SS vessel and heat the entire contents to 95° C., boil it for 30 minutes, start chiller, bring down temperature to 25° C., and dispense the total amount of boiled & cooled DM Water (47.929%) required for the batch into a separate SS vessel and keep it covered. Keep DM Water (2.00%) for rinsing separately in covered SS vessel.
2. Take boiled & cooled DM Water (3.00% in a side vessel, add Citric Acid into it, and mix well to form ‘Phase B’.
3. Charge ‘Phase A’ ingredients in a side SS vessel. Add pre-mixed ‘Phase B’, mix for 5 minutes with continuous stirring at 20 rpm. Stop mixing and heat the entire contents to 70° C. start chiller, bring down temperature to 60° C., maintain at same temperature for 1-2 hrs, till foam disappears completely. Again start chiller and bring down temperature to 35° C. This is ‘Phase AB’.
4. Charge the freshly boiled and cooled ‘Phase D’ DM Water into the main vessel and continue mixing at 20 rpm.
5, Take ‘Phase C’ Glycerin in a side SS vessel and disperse ‘Amaze-XT’ into it by using Karl Homogenizer to form smooth slurry. Scrap occasionally to remove any lumps.
6. Add the pre-mixed slurry of Glycerin and Amaze-XT into the vortex of the water in the main vessel to form ‘Phase CD’, Mix it at very high speed of 80 rpm for 5 minutes, scrap the sides of the vessel and anchor blades, again mix at 80 rpm for 10 minutes.
7. Charge the ‘Phase E’ Potassium Sorbate and boiled & cooled DM water (1.00%) into the side vessel and mix to form a uniform solution.
8. Transfer ‘Phase E’ into main vessel containing Amaze-XT gel to form ‘Phase CDE’, and continue mixing at 60 rpm for 20 minutes to form a uniform gel till there are no lumps found in the solution. Scrap the sides of the vessel and anchor blades, and again mix at 30 rpm for 10 minutes.
9. Start Vacuum at 450-550 mm of Hg with mixing at 30 rpm for 60 minutes till no lumps remain and air bubbles subside to minimum.
10. Under vacuum charge the ‘Phase AB’ into the main vessel containing ‘Phase CDE’, from bottom and mix with continuous stirring at 10 rpm till the complete transfer take place, to form ‘Phase ABCDE’.
11. Charge the ‘Phase F’ Active, in a stainless steel container and mix with Plantacare 818 UP to form slurry.
12, Release vacuum and transfer the pre-mixed Phase F′ to the main vessel by passing it through nylon muslin filter with continuous stirring at 20 rpm for 10 minutes to form ‘Phase ABCDEF’.
13. Give Vacuum at 550 mm of Hg for 60 minutes with continuous mixing at 20 rpm, till all the air bubbles are removed.
14. Charge the ‘Phase G’ ingredients into the side SS vessel and mix for 5 minutes to form a uniform blend of Perfume & Natural Vitamin E.
15. Release vacuum and transfer the pre-mixed ‘Phase G’ into the main vessel to form ‘Phase ABCDEFG’ and mix for 20 minutes.
16. Charge the ‘Phase H’ ingredient, Biovert Substrate Solution to the main vessel and mix with continuous stirring for 5 minutes.
17. Take out 5 Kg of bulk from below from the main vessel in a side SS vessel, add the ‘Phase H’ ingredient, Biovert Enzyme Solution into it, mix well with SS Spatula and transfer the entire contents into the main vessel to form ‘Phase ABCDEFGH’ and mix for 20 minutes.
18. Check the pH of the product, which should be in the range of pH=4.5-5.4.

### Example-9

### Preparation of Extract from Herbal Blend for EGBS-09 by Super Critical Fluid Extraction

The shade dried material of herbal blend of Oryza sativa grains and/or Cicer arietinum seeds and/or Hibiscus rosa-sinensis flowers and/or Vetiver zizanioides roots in the ratio of 1:1:1:1 respectively and in different ratios also was pulverized to coarse powder and about 100 Kg of powdered herbal blend was placed in a SCF extractor at the temperature of 40-50° C. at high pressure of 300-350 bar using carbon dioxide as super critical fluid for extraction up to 4 to 6 hours and then the extract was collected in the collection vessel and evaporated at room temperature to remove any further residues of carbon dioxide, The extract thus obtained was free from any solvent residues and in highest pure form.

### Example-10

### Preparation of Extract from Herbal Blend for EGBS-09 by Aqueous Extraction

The shade dried material of herbal blend of Oryza sativa grains and/or Cicer arietinum seeds and/or Hibiscus rosa-sinesis flowers and/or Vetiver zizanioides roots in the ratio of 1:1:1:1 respectively and in different ratios also was pulverized to coarse powder and about 100 Kg of powdered herbal blend was placed in soxhlator using water as solvent of extraction at refluxing temperature and recycled the process until extraction is completed, then plant extracts were filtered and concentrated to dryness on rotatory evaporator or on steam bath at optimum temperature. The herbal material was also placed in percolator and extracted with water at room temperature for 24 h to 48 h, then plant extracts were filtered and concentrated to dryness on rotatory evaporator or on steam bath at optimum temperature and under reduced pressure.

### Example-11

### Preparation of Extract from Herbal Blend for ERBW-18 by Super Critical Fluid Extraction

The shade dried material of herbal blend of Vetiveria zizanoides roots and/or Cynodon dactylon whole plant and/or Cyperus rotundus roots and/or Sida cordifolia roots and/or Glycyrrhiza glabra roots in the ratio of 1:1:1:1:1 respectively and in different ratios also was pulverized to coarse powder and about 100 Kg of powdered herbal blend was placed in a SCF extractor at the temperature of 40-50 degree C. at high pressure of 300-350 bar using carbon dioxide as super critical fluid for extraction up to 4 to 6 hours and then the extract was collected in the collection vessel and evaporated at room temperature to remove any further residues of carbon dioxide. The extract thus obtained was free from any solvent residues and in highest pure form.

### Example-12

### Preparation of Extract from Herbal Blend for ERBW-18 by Aqueous Extraction

The shade dried material of herbal blend of Vetiveria zizanoides roots and/or Cynodon dactylon whole plant and/or Cyperus rotundus roots and/or Sida cordifolia roots and/or Glycyrrhiza glabra roots of in the ratio of 1:1:1:1:1 respectively and in different ratios also was pulverized to coarse powder and about 100 Kg of powdered herbal blend was placed in soxlator using water as solvent of extraction at refluxing temperature and recycled the process until extraction is completed, then plant extracts were filtered and concentrated to dryness on rotatory evaporator or on steam bath at optimum temperature. The herbal material was also placed in percolator and extracted with water at room temperature for 24 h to 48 h, then plant extracts were filtered and concentrated to dryness on rotatory evaporator or on steam bath at optimum temperature and under reduced pressure.

### Example-13

### Preparation of Extract from Herbal Blend for EEBW-27 by Super Critical Fluid Extraction

The shade dried material of herbal blend of Aegle marmelos Fruits and/or Myristica fragrans nutmeg and/or Carica papaya fruits and/or Ananas sativus fruits in the ratio of 1:1:1:1 respectively and in different ratios also was pulverized to coarse powder and about 100 Kg of powdered herbal blend was placed in a SCF extractor at the temperature of 40-50 C at high pressure of 300-350 bar using carbon dioxide as super critical fluid for extraction up to 4 to 6 hours and then the extract was collected in the collection vessel and evaporated at room temperature to remove any further residues of carbon dioxide. The extract thus obtained was free from any solvent residues and in highest pure form.

### Example-14

### Preparation of Extract from Herbal Blend for EEBW-27 by Aqueous Extraction

The shade dried material of herbal blend of Aegle marmelos fruits and/or Myristica fragans nutmeg and/or Carica papaya fruits and/or Ananas sativus fruits in the ratio of 1:1:1:1 respectively and in different ratios also was pulverized to coarse powder and about 100 Kg of powdered herbal blend was placed in soxhlator using water as solvent of extraction at refluxing temperature and recycled the process until extraction is completed, then plant extracts were filtered and concentrated to dryness on rotatory evaporator or on steam bath at optimum temperature. The herbal material was also placed in percolator and extracted with water at room temperature for 24 h to 48 h, then plant extracts were filtered and concentrated to dryness on rotatory evaporator or on steam bath at optimum temperature and under reduced pressure.

### Example-15

### Preparation of Extract from Herbal Blend for EPBW-36 by Super Critical Fluid Extraction

The shade dried material of herbal blend of Melia azadirachta Leaves and/or Curcuma longa roots and/or Terminalia chebula fruits and/or Terminalia bellerica fruits and/or Phyllanthus emblica fruits and/or Ficus racemosa bark and/or Ficus bengalensis bark and/or Ficus religiosa bark in the ratio of 1:1:1:1:1:1:1:1 respectively and in different ratios also was pulverized to coarse powder and about 100 Kg of powdered herbal blend was placed in a SCF extractor at the temperature of 40-50 C at high pressure of 300-350 bar using carbon dioxide as super critical fluid for extraction up to 4 to 6 hours and then the extract was collected in the collection vessel and evaporated at room temperature to remove any further residues of carbon dioxide. The extract thus obtained was free from any solvent residues and in highest pure form.

### Example-16

### Preparation of Extract from Herbal Blend for EPBW-36 by Aqueous Extraction

The shade dried material of herbal blend of Melia azadirachta leaves and/or Curcuma longa roots and/or Terminalia chebula fruits and/or Terminalia bellerica fruits and/or Phyllanthus emblica fruits and/or Ficus racemosa bark and/or Ficus bengalensis bark and/or Ficus religiosa bark in the ratio of 1:1:1:1:1:1:1:1 in the ratio of 1:1:1:1 respectively and in different ratios also was pulverized to coarse powder and about 100 Kg of powdered herbal blend was placed in soxhlator using water as solvent of extraction at refluxing temperature and recycled the process until extraction is completed, then plant extracts were filtered and concentrated to dryness on rotatory evaporator or on steam bath at optimum temperature. The herbal material was also placed in percolator and extracted with water at room temperature for 24 h to 48 h, then plant extracts were filtered and concentrated to dryness on rotatory evaporator or on steam bath at optimum temperature and under reduced pressure.

### Example-17

### Conditioning Evaluation

1. Hair tress/Hair Swatch In-vitro evaluation.
2. Chemically untreated human hairs were selected for evaluation that have no permanent waves, no hair color or even beat up from styling with curling irons.
3. Approximately 20 cm long and 15 g weight hair swatches were selected.
4. 1.5 gram of shampoo was taken for wash and hair swatches were washed in manually and rinsed in the running water.
5. Cold air blowers dried up the hair swatches.
6. The hair swatches were kept in a stand.
7. Trained panel members evaluated the hair swatches.
8. They were asked to rank the product in 10 scales.
9. The ranking scale was given below

| | | | | | | | | | |
|---|---|---|---|---|---|---|---|---|---|
| 1 | 2 | 3 | 4 | 5 | 6 | 7 | 8 | 9 | 10 |

| | | |
|---|---|---|
| Not acceptable | Acceptable | Excellent |

### Example-18

### Results of the Evaluation

Different gums, that is, guar gum, xanthan gum & Amaze-XT, was evaluated, was evaluated at same level at 0.85%, in formula A, B & C respectively, Hair swatches were made like the above protocol and the results are below.

The averaged ratings are

| Parameters | Formula A | Formula B | Formula C |
|---|---|---|---|
| In use feel | 5 | 4 | 8 |
| Wet combing | 4 | 4 | 7.5 |
| Dry combing | 5 | 5.5 | 8 |
| Softness of hair | 5.5 | 6 | 9 |

Result:
The formula C, which is with 0.85% Amaze-XT excels out Formula A and Formula B with Guar Gum & Xanthan Gum, respectively.

### Example-19

A Clinical evaluation of gentle baby shampoo was carried out in 15 subjects during April-2009 to June-2009.

Following parameters were evaluated on visual analogue scale of 0-3. (0-Unpleasant, 1-Neutral, 2-Good, 3-Excellent)

### 1. Fragrance

### 2. Ease of Application

3. Feel after Application

### 4. Ease of Removal

Evaluation of following parameters was done after using gentle baby shampoo on a visual analogue scale of 0-4.

1 Cleansing efficacy
2 Moisturising effect
3 Feel of freshness

### Example-20

### Results of Clinical Evaluation

1 All the 15 subjects completed the study protocol and none of them withdrew from the study.
2 Subjects compliance was very good.
3 None of subjects exhibited any adverse effects during the study period.
4 Evaluation of effects was as follows

| | | Findings/ |
|---|---|---|
| | | satisfaction |
| Number | Parameter | (Mean) |
| 1 | Fragrance | 3.00 |
| 2 | Ease of application | 2.73 |
| 3 | Feel after application | 2.66 |
| 4 | Ease of removal | 2.67 |
| 5 | Cleansing efficacy | 3.46 |
| 6 | Moisturising effect | 3.8 |
| 7 | Feel of freshness | 3.6 |

Adverse effects profile (Dermal safety evaluation)

Application: Twice weekly

Number of subjects: 15

Duration of application: 2 weeks.

| Type of adverse | On first | | |
|---|---|---|---|
| effect | application | Week 1 | Week 2 |
| Irritation | Nil | Nil | Nil |
| Erythema | Nil | Nil | Nil |
| Prurutis | Nil | Nil | Nil |
| & Urticaria | | | |
| Dryness & | Nil | Nil | Nil |
| Stinging | | | |

While this invention has been described in detail with reference to certain preferred embodiments, it should be appreciated that the present invention is not limited to those precise embodiments. Rather, in view of the present disclosure, which describes the current best mode for practicing the invention, many modifications and variations would present themselves to those skilled in the art without departing from the scope and spirit of this invention.

1.-11. (canceled)

12. A formulation for preparing cleansing compositions of optimum viscosity, comprising dehydroxanthan gum and alkylpolyglucosides.

13. The formulation according to claim 12, wherein dehydroxanthan gum is in the range of 0-5%.

14. The formulation according to claim 12, wherein alkylpolyglucoside is in the range of from 5-50%.

15. A herbal cleansing composition comprising extracts of herbs along with dehydroxanthan gum and alkylpolyglucoside and cosmeceutically acceptable excipients, wherein the herbal extracts are prepared employing a super critical fluid extraction and/or water extraction.

16. The composition according to claim 15, wherein the extract of herbs is obtained from a blend of herbs selected from Oryiza sativa, Cicer arietinum, Hibiscus rosa-sinensis, Vetiver zizanioides, Cynodon dactylon, Cyperus rotundus, Sida cordifolia, Glycyrrhiza glabra, Aegle marmelos, Myristica fragrans, Carica papaya, Ananas sativus, Melia azadirachta, Curcuma longa, Terminalia chebula, Terminalia bellerica, Phyllanthus emblica, Ficus racemosa, Ficus bengalensis, Ficus religiosa or in various combinations thereof.

17. The composition according to claim 15, wherein the blend extract is used in the range of 0.0001% to 20.00%.

18. The composition according to claim 15, wherein said composition is formulated in shampoos and face wash formulation.

19. The composition according to claim 18, wherein shampoo is Gentle Baby Shampoo.

20. The composition according to claim 18, wherein face wash is revitalising body wash, exfoliating body wash and purifying body wash.

21. The composition according to claim 20, wherein the Revitalizing body wash comprising a herbal blend extract of herbs Vetiveria zizanoides, Cynodon dactylon, Cyperus rotundus, Sida cordifolia, Glycyrrhiza glabra along with dehydroxanthan gum and alkylpolyglucoside and cosmeceutically acceptable excipients.

22. The composition according to claim 20, wherein the exfoliating body wash comprising a herbal blend extract of herbs Aegle marmelos, Myristica fragrans, Carica papaya, Ananas sativus along with dehydroxanthan gum and alkylpolyglucoside and cosmeceutically acceptable excipients.

23. The composition according to claim 20, wherein the purifying body wash comprising a herbal blend extract of herbs Melia azadirachta, Curcuma longa, Terminalia chebula, Terminalia bellerica, Phyllanthus emblica, Ficus racemosa, Ficus bengalensis, Ficus religiosa along with dehydroxanthan gum and alkylpolyglucoside and cosmeceutically acceptable excipients.

24. The composition according to claim 15, wherein said composition is prepared by a method comprising blending the herbs and extracting the resultant herbal blend employing SCFE, mixing the resultant extract of blend with dehydroxanthan gum and alkylpolyglucoside and cosmeceutically acceptable excipients.

25. The composition according to claim 15, wherein said composition is prepared by a method comprising blending the herbs and extracting the resultant herbal blend employing percolation or soxhalation using water, mixing the resultant extract of blend with dehydroxanthan gum and alkylpolyglucoside and cosmeceutically acceptable excipients.
